(19) United States
(12) Patent Application Publication
(10) Pub. No.: US 2025/0114634 A1
(43) Pub. Date: Apr. 10, 2025

(54) TRANSCRANIAL IMPLANT FOR DELIVERY OF PHOTODYNAMIC THERAPY

(71) Applicants: The Board of Trustees of the University of Illinois, Urbana, IL (US); Carle Foundation Hospital, Urbana, IL (US)

(72) Inventors: Anant Naik, Eagan, MN (US); Emily Smith, North Mankato, MN (US); Alexander Smith, Urbana, IL (US); Brian Diner, Champaign, IL (US); Mohamed A. Belakhoua, Urbana, IL (US); Jack Stender, Chicago, IL (US)

(21) Appl. No.: 18/908,427

(22) Filed: Oct. 7, 2024

Related U.S. Application Data
(60) Provisional application No. 63/588,682, filed on Oct. 6, 2023.

(51) Int. Cl. A61N 5/06; A61F 2/28

(52) U.S. Cl. CPC A61N 5/0622; A61F 2/2875; A61N 5/0603; A61N 5/062; A61N 2005/0626; A61N 2005/0632; A61N 2005/0651

(57) ABSTRACT

A transcranial implant is used to deliver photodynamic therapy. The transcranial implant may include a frame to be coupled to a skull of a patient, a light source coupled to the frame, and a controller coupled to the frame and in communication with the light source. The controller controls the light source to deliver light to a resection margin-containing region within the patient.

### CROSS-REFERENCE TO RELATED APPLICATIONS

This application claims the benefit of U.S. Provisional Patent Application Ser. No. 63/588,682, filed on Oct. 6, 2023, and entitled “TRANSCRANIAL IMPLANT FOR DELIVERY OF PHOTODYNAMIC THERAPY,” which is herein incorporated by reference in its entirety.

### BACKGROUND

Approximately 15,000 patients are diagnosed with glioblastomas annually. Each patient has a grim prognosis, with a median survival of 12-18 months, and near-universal recurrence despite maximal surgical resection. The reason why this occurs is that despite aggressive care, glioblastoma cancer cells disperse throughout the brain beyond the surgical margin of resection. Photodynamic therapy (PDT), in conjunction with the drug 5-ALA, offers a treatment to induce free radical damage beyond the surgical margin to destroy residual tumor cells. This therapy is offered through a large laser that can only be used intraoperatively. However, this one-time therapy expands survival from 12 months to 28 months in published trials.

### SUMMARY OF THE DISCLOSURE

It is an aspect of the present disclosure to provide a transcranial implant for delivering photodynamic therapy. The transcranial implant includes a frame to be coupled to a skull of a patient, a light source coupled to the frame, and a controller coupled to the frame and in communication with the light source. The controller controls the light source to deliver light to a resection margin-containing region within the skull of the patient.

It is another aspect of the present disclosure to provide a method for delivering photodynamic therapy to a patient. The method includes administering a fluorescent agent to a patient, where the fluorescent agent is taken up by a tumor resection margin in the patient; arranging a transcranial implant adjacent the tumor resection margin, where the transcranial implant houses a light source; and operating the light source of the transcranial implant to deliver light to the tumor resection margin to irradiate the fluorescent agent taken up by the tumor resection margin, thereby generating free radicals in the tumor resection margin to effectuate photodynamic therapy of the tumor resection margin.

### BRIEF DESCRIPTION OF THE DRAWINGS

FIG. 1 shows an example transcranial photodynamic therapy (PDT) device.

FIG. 2 shows an example of a transcranial implant for supporting the operative components of the transcranial PDT device.

FIG. 3 shows an example of a transcranial implant coupled to a skull according to some embodiments described in the present disclosure.

FIG. 4 shows a block diagram of example of components that can implement the transcranial PDT device.

FIG. 5 is a flowchart setting forth the steps of an example method for providing photodynamic therapy using a transcranial PDT device according to some embodiments described in the present disclosure.

### DETAILED DESCRIPTION

Described here are systems and methods for photodynamic therapy via an transcranial implant. Advantageously, the systems and methods described in the present disclosure can be used as an adjuvant therapy beyond the standard of care to prevent recurrence of cancerous tumors, such as lobar glioblastoma tumors. The disclosed systems and methods are capable of repeated photodynamic for superficial tumors, thereby increasing the survival benefit to brain cancer patients.

In general, photodynamic therapy uses fluorescent agents that are selectively, or otherwise preferentially, taken up by a targeted tumor. As one non-limiting example, the fluorescent agent may be 5-aminolevulinic acid (5-ALA). Applying a light source to the tumor following uptake of the fluorescent agent generates free radicals that, in turn, cause damage to the tumor tissues.

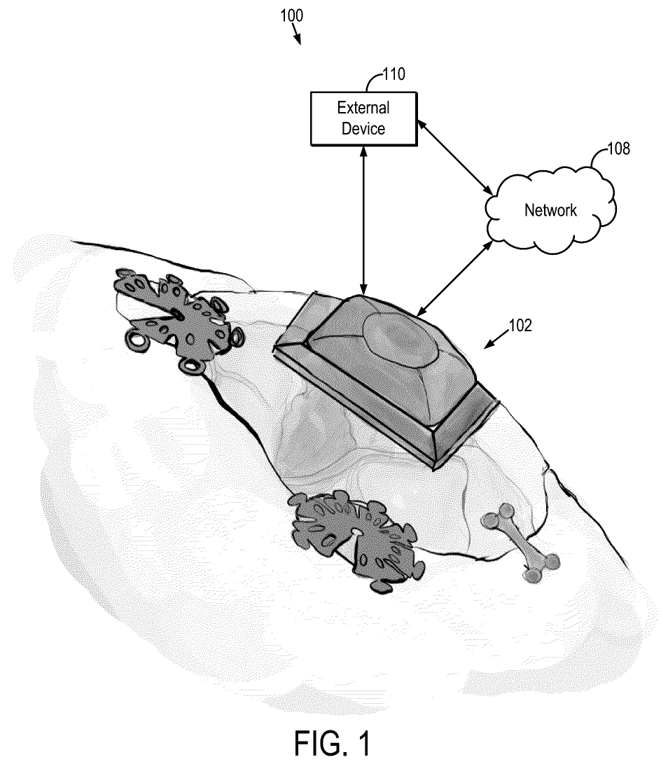

It is an aspect of the present disclosure to provide an transcranial implant for delivering photodynamic therapy. FIG. 1 illustrates an example of such an transcranial photodynamic therapy (PDT) device 100. The transcranial PDT device 100 may be implanted into a subject's skull following tumor resection. In general, the transcranial PDT device 100 includes a transcranial implant 102 that houses a light source 104 for providing post-operative PDT to the subject. Advantageously, using the transcranial PDT device 100 described in the present disclosure, there is no need to reopen the cranial window for PDT delivery, unlike existing techniques for PDT, which require an intraoperative delivery of PDT.

The transcranial PDT device may also include, or be in communication with, one or more of a network 108, an external device 110, and/or another computer system such as a server. An external device 110 may be used to communicate with the transcranial PDT device 100, such as to send data (e.g., instructions, firmware updates) to the controller 120 and/or to receive data (e.g., sensor data) from the transcranial PDT device 100. These collected or stored data can be transmitted to the external device 110 from the transcranial PDT device 100 via a network 108, which may be a wired network or a wireless network.

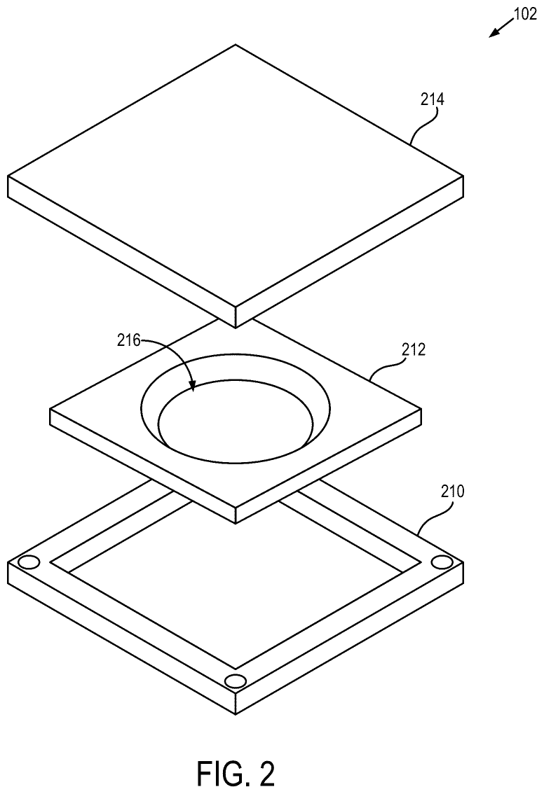

As shown in FIG. 2, the transcranial implant 102 may include a frame 210 that is coupled to a skull of a patient. A sheath 212 is coupled to the frame 210. The operative components of the transcranial PDT device 100 are coupled to the sheath, such that the transcranial PDT device 100 is made to be effectively coupled to the patient. In this way, the light source 104 is arranged and held in position with respect to a tumor, tumor containing region, resection margin, and/or resection margin containing region within the patient. The transcranial implant 102 may also include a cover 214 that can be removably coupled to the frame 210. In some constructions, the sheath 212 is arranged between and enclosed within the frame 210 and the cover 214. The cover 214 may be a removable cover that is coupled to the frame 210 and is sized such that when the sheath 212 and cover 214 are both coupled to the frame 210 the sheath 212 will be arranged between and enclosed within the frame 210 and the cover 214. For instance, after the sheath 212 is arranged within the frame 210, the cover 214 may be coupled to the frame 210 to protect the operative components of the transcranial PDT device 100 and to provide a barrier between the patient's internal anatomy and the surrounding environment.

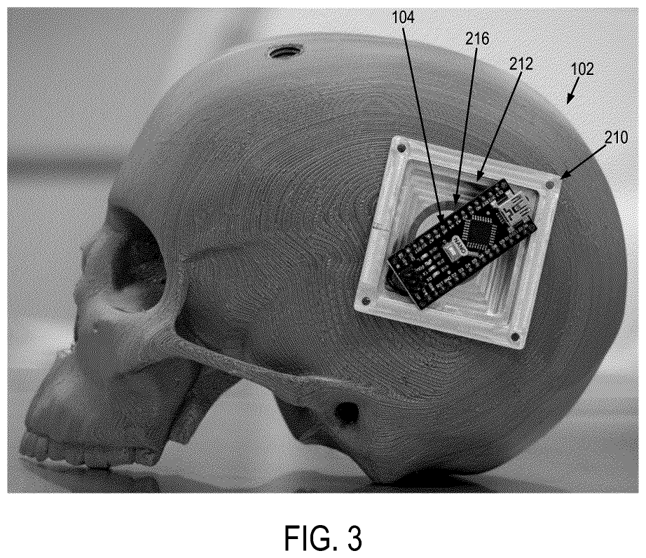

In the illustrated example, the frame 210 may generally include a square, rectangular, or other polygonal shaped frame for receiving the sheath 212 and/or cover 214. The frame 210 may be coupled to the patient, as illustrated in FIGS. 1 and 3. For example, the frame 210 may be affixed to a skull of the patient, or may otherwise be affixed or coupled to a skin surface of the patient, or to another anatomical surface of the patient. The frame 210 may be affixed or otherwise coupled to the patient via one or more fasteners (e.g., bone screws), adhesives, via suction or a vacuum seal, or the like. The frame 210 may be composed of a suitable material for coupling the frame 210 to the patient while providing structural support for the operative components of the transcranial PDT device 100. In general, the frame 210 is composed of a biocompatible material. As one non-limiting example, the frame 210 may be composed of a metallic material, such as a metal or metal alloy. For instance, the frame 210 may be composed of machined aluminum or the like. In other examples, the frame 210 may be composed of a non-metallic material, such as a polymer or plastic. In some instances, the frame 210 may be manufactured from a non-metallic material using an additive manufacturing technique, such as 3D printing.

As noted above, the operative components of the transcranial PDT device 100 are coupled to the sheath 212, such that the transcranial PDT device 100 is made to be effectively coupled to the patient. As a non-limiting example, the sheath 212 can include a central aperture 216 that is sized to receive the operative components of the transcranial PDT device 100. For instance, the central aperture 216 can receive the light source 104 to provide an optical path for the light emitted from the light source 104.

In some embodiments, the light source 104 may be securely held in place by coupling the light source 104 and/or other operative components to the sheath 212 or the frame 210. For example, as illustrated in FIG. 3, the light source 104 and/or controller 120 can be coupled to the sheath 212, which is received and retained by the frame 210. In still other embodiments, the sheath 212 may be integral with the frame 210, such as by machining the frame 210 and the sheath 212 as a single integral component.

The cover 214 may attach to the frame 210 via an interference fit, a snap fit, a hinged connection, a threaded connection, or the like. The cover 214 can be constructed of a suitable material for protecting the operative components of the transcranial PDT device 100, such as a metallic material (e.g, a metal, a metal alloy) or a non-metallic material (e.g., a polymer, a plastic, or the like). As a non-limiting example, the cover 214 can be composed of machined aluminum. In some instances, the cover 214 can be composed of an optically transparent or translucent material, such that the operative components of the transcranial PDT device 100 can be observed during the delivery of PDT to the patient. In other instances, it may be advantageous to construct the cover 214 from an optically opaque material so as to reduce or otherwise eliminate light leakage into, or from, the transcranial implant 102.

The network 108 may be a long-range wireless network such as the Internet, a local area network (LAN), a wide area network (WAN), or a combination thereof. In other embodiments, the network 108 may be a short-range wireless communication network, and in yet other embodiments, the network 108 may be a wired network using, for example, USB cables. Additionally or alternatively, the network 108 may include a combination of long-range, short-range, and/or wired connections. In some embodiments, the network 108 may include both wired and wireless devices and connections. Similarly, a server may transmit information to the external device 110 to be forwarded to the transcranial PDT device 100.

In some embodiments, the transcranial PDT device 100 communicates directly with the external device 110. For example, the transcranial PDT device 100 can transmit data and settings to the external device 110. Similarly, the transcranial PDT device 100 can receive data (e.g., settings, firmware updates, etc.) from the external device 110.

In some other embodiments, the transcranial PDT device 100 may bypass the external device 110 to access the network 108 and communicate with a server or other computer system via the network 108. In some embodiments, the transcranial PDT device 100 is equipped with a long-range transceiver instead of or in addition to a short-range transceiver. In such embodiments, the transcranial PDT device 100 may communicate directly with a server via the network 108 (in either case, bypassing the external device 110). In some embodiments, the transcranial PDT device 100 may communicate directly with both a server and the external device 110. In such embodiments, the external device 110 may, for example, generate a graphical user interface to facilitate control and programming of the transcranial PDT device 100, while the server may store and analyze larger amounts of data for future programming or operation of the transcranial PDT device 100. In other embodiments, however, the transcranial PDT device 100 may communicate directly with the server without utilizing a short-range communication protocol with the external device 110.

In the illustrated embodiment, the transcranial PDT device 100 communicates with the external device 110. The external device 110 may include, for example, a smartphone, a tablet computer, a cellular phone, a laptop computer, a smartwatch, and the like. The transcranial PDT device 100 communicates with the external device 110, for example, to transmit at least a portion of data received by the transcranial PDT device 100. Additionally, the transcranial PDT device 100 may receive data from the external device 110. For instance, a user may send instructions to control operation of the transcranial PDT device 100 via the external device 110. In these instances, the external device 110 may control operation of the light source 104 (e.g., turning the light source 104 on and off to deliver photodynamic therapy), may control delivery of a fluorescent agent to a tumor, and so on.

In some embodiments, the external device 110 may include a short-range transceiver to communicate with the transcranial PDT device 100, and a long-range transceiver to communicate with a server. In the illustrated embodiment, transcranial PDT device 100 can also include a transceiver to communicate with the external device 110 via, for example, a short-range communication protocol such as Bluetooth® or Wi-Fi®. In some embodiments, the external device 110 bridges the communication between the transcranial PDT device 100 and a server. For example, the transcranial PDT device 100 may transmit data to the external device 110, and the external device 110 may forward the data from the transcranial PDT device 100 to a server over the network 108.

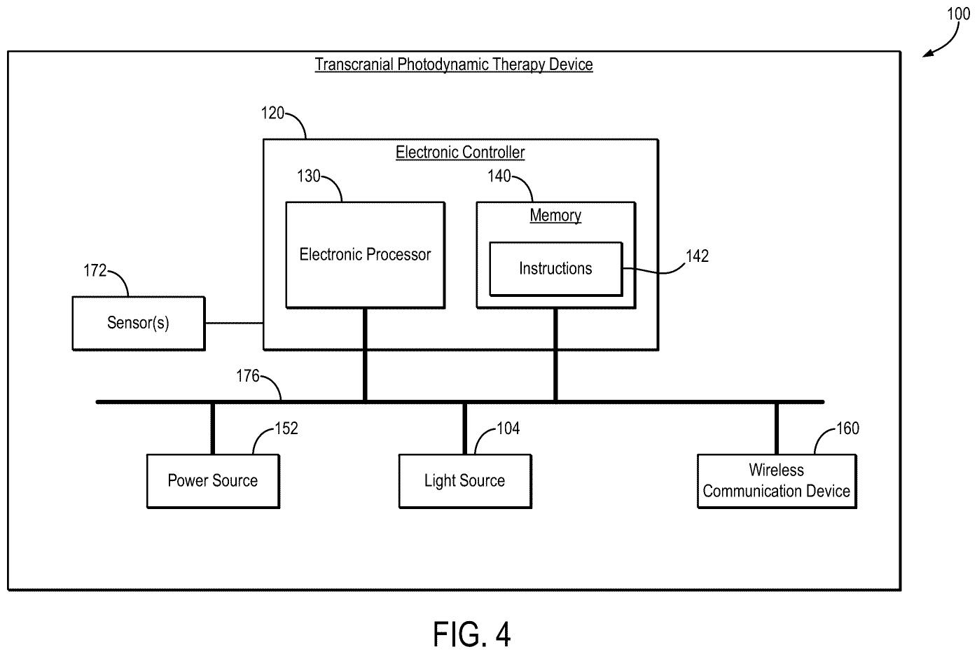

FIG. 4 shows a block diagram of an example transcranial PDT device 100. The transcranial PDT device 100 includes an electronic controller 120, a light source 104, a power source 152 (e.g., a battery pack, a portable power supply), etc. In the illustrated embodiment, the transcranial PDT device also includes a wireless communication device 260 and one or more sensors 172. In some embodiments, the components of the transcranial PDT device 100 shown in FIG. 3 may be coupled to one or more printed circuit boards, which may be arranged within the sheath 212 of the transcranial implant 102 part of the transcranial PDT device 100.

The transcranial PDT device 100 receives electrical power from a power source 152, which supplies power for operating the controller 120, the light source 104, wireless communication device 160, and other electronic components of the transcranial PDT device 100. In some examples, the power source 152 may be a battery, which in some instances may be a rechargeable battery.

The light source 104 may include a light emitting diode (LED), a laser light source, or the like. As one non-limiting example, the light source 104 may be an LED light source that includes one or more LEDs. In some instances, the light source 104 may include one or more 130 mW LEDs. In some other instances, the light source 104 may include a laser light source. Additionally, the light source 104 may include a light diffuser optically coupled to the light source.

The light source 104 may emit light at one or more wavelengths. As one example, the light source 104 may emit light at a wavelength of 600 nm. In other embodiments, the light source 104 may be configured to emit light at other wavelengths, at bandwidths containing more than one wavelength of light, or the like. In general, the wavelength(s) of light emitted by the light source 104 will be selected based on the fluorescent agent to be administered to the patient. Advantageously, the wavelength of light can be selected to provide therapeutic photoirradiation of the fluorescent agent. Thus, in some embodiments, the light source 104 may include one or more light sources capable of emitting light at different wavelengths. In this configuration, the controller 120 can control operation of the light source(s) 104 to emit light at the effective wavelength for providing photodynamic therapy based on the fluorescent agent that has been administered to the patient.

The controller 120 can include an electronic processor 130 and memory 140. The electronic processor 130 and memory 140 can communicate over one or more control buses, data buses, etc., which can include a device communication bus 176. The control and/or data buses are shown generally in FIG. 1 for illustrative purposes. The use of one or more control and/or data buses for the interconnection between and communication among the various modules, circuits, and components would be known to a person skilled in the art.

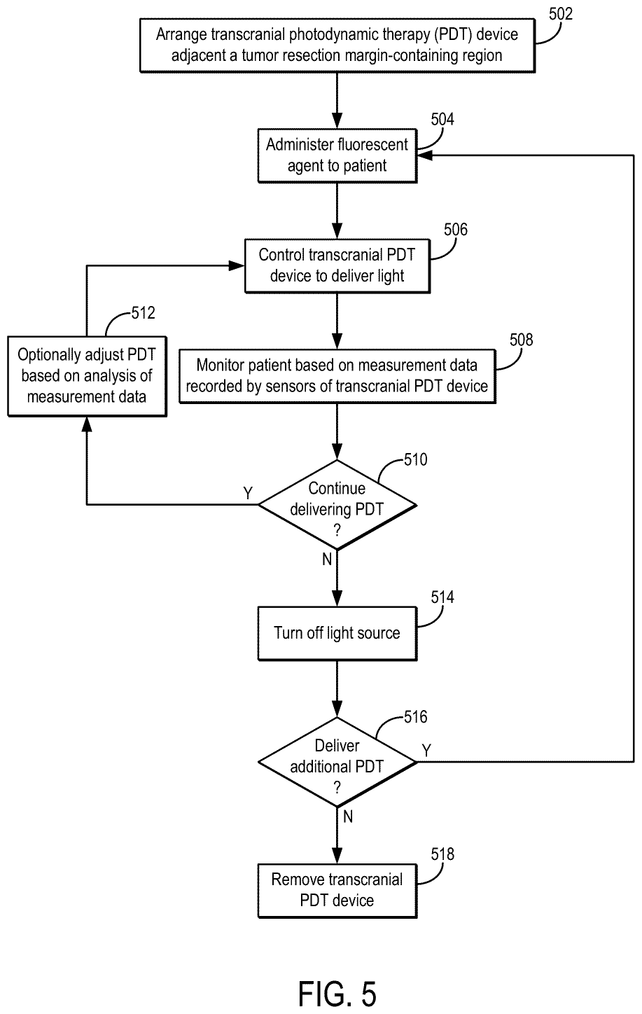

The electronic processor 130 can be configured to communicate with the memory 140 to store data and retrieve stored data. The electronic processor 130 can be configured to receive instructions 142 and data from the memory 140 and execute, among other things, the instructions 142. In particular, the electronic processor 130 executes instructions 142 stored in the memory 140. Thus, the electronic controller 120 coupled with the electronic processor 130 and the memory 140 can be configured to perform the methods described herein (e.g., some or all of the steps of the method illustrated in FIG. 5).

The memory 140 can include read-only memory (ROM), random access memory (RAM), other non-transitory computer-readable media, or a combination thereof. The memory 140 can include instructions 142 for the electronic processor 130 to execute. The instructions 142 can include software executable by the electronic processor 130 to enable the electronic controller 120 to, among other things, receive data and/or commands, transmit data, control operation of the light source 104, and the like. The software can include, for example, firmware, one or more applications, program data, filters, rules, one or more program modules, and other executable instructions.

The electronic processor 130 is configured to retrieve from memory 140 and execute, among other things, instructions 142 related to the control processes and methods described herein. The electronic processor 130 is also configured to store data on the memory 140 including sensor data acquired with one or more sensors 172.

As illustrated, the transcranial PDT device 100 includes a wireless communication device 160. The wireless communication device 160 is coupled to the electronic controller 120 (e.g., via the device communication bus 176). The wireless communication device 160 may include, for example, a radio transceiver and antenna, a memory, and an electronic processor. The radio transceiver and antenna operate together to send and receive wireless messages to and from the external device 110, an additional computer system or server, and/or the electronic processor of the wireless communication device 160. The memory of the wireless communication device 160 stores instructions to be implemented by the electronic processor and/or may store data related to communications between transcranial PDT device 100 and the external device 110, one or more additional computer systems, and/or a server.

The electronic processor for the wireless communication device 160 controls wireless communications between the transcranial PDT device 100 and the external device 110, one or more additional computer systems, and/or a server. For example, the electronic processor of the wireless communication device 160 buffers incoming and/or outgoing data, communicates with the electronic processor 130 and determines the communication protocol and/or settings to use in wireless communications.

In some embodiments, the wireless communication device 160 is a Bluetooth® controller. The Bluetooth® controller communicates with the external device 110, one or more additional computer systems, and/or a server employing the Bluetooth® protocol. In such embodiments, therefore, the external device 110, one or more additional computer systems, and/or the server and the transcranial PDT device 100 are within a communication range (i.e., in proximity) of each other while they exchange data. In other embodiments, the wireless communication device 160 may communicate using other protocols (e.g., Wi-Fi, cellular protocols, a proprietary protocol, etc.) over a different type of wireless network. For example, the wireless communication device 160 may be configured to communicate via Wi-Fi through a wide area network such as the Internet or a local area network, or to communicate through a piconet (e.g., using infrared or NFC communications). The communication via the wireless communication device 160 may be encrypted to protect the data exchanged between the transcranial PDT device 100 and the external device 110, one or more additional computer systems, and/or the server from third parties.

The wireless communication device 160, in some embodiments, exports sensor data from the transcranial PDT device 100 (e.g., from the electronic processor 130), as described above. For instance, the wireless communication device 160 may export measurement data of one or more physiological parameters (e.g., temperature, blood flow, electrophysiological signals) measured by sensors 172 contained within or otherwise coupled to the transcranial PDT device 100.

The wireless communication device 160 may also enable the transcranial PDT device 100 to sync to or otherwise communicate data with other devices, such as an external device 110 that is configured as a smartwatch or other wearable device. In some instances, the transcranial PDT device 100 can send and receive health information for the user (e.g., by syncing with a heartrate monitor, smart watch, or other wearable device).

In some embodiments, the electronic controller 120 is also connected to one or more sensors 172, which may include electrophysiological sensors (e.g., electrodes), temperature sensors or temperature sensing circuits, blood flow monitors, inertial sensors or inertial sensing circuits (e.g., accelerometers, gyroscopes, magnetometers), pressure sensors or pressure sensing circuits (e.g., a barometer), or the like. The transcranial PDT device 100 may also include connections (e.g., wired or wireless connections) for external sensors. The sensor(s) 172 can transmit their data to external sources, such as an external device 110 and/or a server (e.g., via the network 108 or directly).

Referring now to FIG. 5, a flowchart is illustrated as setting forth the steps of an example method for photodynamic therapy of a tumor using an transcranial implant.

The method includes, following a tumor resection procedure, arranging a transcranial photodynamic therapy device adjacent a region from which the tumor was resected from a patient, as indicated at step 502. For instance, the transcranial implant 102 may be coupled to the patient's skull (e.g., by coupling the frame 210 to the skull) and then coupling the remainder of the transcranial PDT device 100 to the patient's skull (e.g., via coupling the sheath 212 to the frame 210 of the transcranial implant 102).

A fluorescent agent is then administered to the patient, as indicated at step 504. The fluorescent agent may be administered to the subject in a number of ways. As one example, the fluorescent agent may be administered by an intratumoral delivery, such as by injecting the fluorescent agent directly into the tumor margin. As another example, the fluorescent agent may be administered via an intranasal delivery. Alternatively, the fluorescent agent may be administered systemically to the subject. In these instanced, the fluorescent agent may be introduced into a brain tumor by transient disruption of the blood-brain barrier, such as by using focused ultrasound or other means for transiently disrupting the blood-brain barrier. Systemic delivery of the fluorescent agent may also be facilitated by cell carriers, such as nanoparticles, nanotubes, nanocrystals, liposomes, viral carriers, and so on.

In some embodiments, the fluorescent agent may be administered via the transcranial PDT device 100. For example, the transcranial PDT device 100 may include a reservoir or other container holding the fluorescent agent, which may then be administer from the reservoir or other container by controlling the transcranial PDT device 100.

After the fluorescent agent has been administered to the patient, the transcranial PDT device 100 is controlled to turn on the light source 104, as indicated at step 506. The light source 104 may be operated in a continuous mode, a pulsed mode, or otherwise by turning the light source 104 on and off according to a planned pattern or pulse sequence.

In some examples, while photodynamic therapy is being provided to the patient, one or more sensors (e.g., sensors 172) may be operated to acquire measurement data from the patient, as indicated at step 508. These measurement data may be processed (e.g., by an external device 110, server, or other computer system) to monitor the efficacy of the PDT, identify the presence of tumorous tissue in the resection margin, and so on. Treatment can proceed by delivering additional light into the tumor resection margin (e.g., a tumor resection margin containing region) as desired or based on feedback from analysis of the measurement data, or until a predetermined amount of therapy has been administered, as determined at decision block 510. For example, in general, the timing of treatment may be determined based on patient characteristics, established clinical protocols, and/or feedback from clinical trials. That is, a therapy plan can be determined for the patient before delivering PDT, and the PDT can then be delivered according to that plan. In some instances, based on the analysis of the measurement data the operation of the light source 104 may be modified, such as by adjusting one or more parameters of the pulse sequence used to control delivery of light by the light source 104 (e.g., by adjusting power, duration, etc.), as indicated at step 512.

When treatment is completed, the transcranial PDT device 100 may be turned off, as indicated at step 514. The patient may continue to be monitored (e.g., using the sensors 172 of the transcranial PDT device 100 or otherwise) and additional PDT may be provided according to a treatment plan or until no more cancerous tissues are detected in the resection margin, as determined at decision block 516. When treatment is concluded, the transcranial PDT device 100 may be removed from the patient, as indicated at step 518.

The present disclosure has described one or more preferred embodiments, and it should be appreciated that many equivalents, alternatives, variations, and modifications, aside from those expressly stated, are possible and within the scope of the invention.

1. A transcranial implant for delivering photodynamic therapy, comprising:
a frame to be coupled to a skull of a patient;
a light source coupled to the frame;
a controller coupled to the frame and in communication with the light source to control the light source to deliver light to a resection margin containing region within the patient.

2. The transcranial implant of claim 1, wherein the light comprises at least one light emitting diode.

3. The transcranial implant of claim 2, wherein the at least one light emitting diode comprises a 130 mW light emitting diode.

4. The transcranial implant of claim 1, further comprises a light diffuser optically coupled to the light source.

5. The transcranial implant of claim 1, further comprising a sheath that is coupled to the frame, wherein the light source and controller are both coupled to the sheath, thereby coupling the light source and the controller to the frame.

6. The transcranial implant of claim 5, wherein the sheath is removably coupled to the frame, thereby removably coupling the light source and the controller to the frame.

7. The transcranial implant of claim 5, wherein the sheath is integrally formed with the frame.

8. The transcranial implant of claim 5, further comprising a cover that is removably coupled to the frame and is sized such that when the sheath and cover are both coupled to the frame the sheath is arranged between and enclosed within the frame and the cover.

9. The transcranial implant of claim 5, wherein the sheath includes a central aperture to permit optical transmission of the light from the light source to the resection margin containing region within the patient.

10. The transcranial implant of claim 1, further comprising a sensor in communication with the controller, wherein the sensor acquires measurement data from the patient.

11. The transcranial implant of claim 10, wherein the sensor comprises at least one of an electrophysiological sensor, temperature sensor, a blood flow monitor, an inertial sensor, or a pressure sensor.

12. The transcranial implant of claim 10, wherein the sensor comprises a plurality of sensors and the plurality of sensors comprise at least two sensors selected from the group consisting of an electrophysiological sensors, temperature sensors, blood flow monitors, inertial sensors, or pressure sensors.

13. A method for delivering photodynamic therapy to a patient, the method comprising:
administering a fluorescent agent to a patient, wherein the fluorescent agent is taken up by a tumor resection margin in the patient;
delivering light, by a light source, to the tumor resection margin to irradiate the fluorescent agent taken up by the tumor resection margin, thereby generating free radicals in the tumor resection margin to effectuate photodynamic therapy of the tumor resection margin.

14. The method of claim 13, wherein the fluorescent agent comprises 5-aminolevulinic acid (5-ALA).

15. The method of claim 13, further comprising, while delivering the light to the tumor resection margin, monitoring the tumor resection margin by acquiring measurement data from at least one sensor.

16. The method of claim 15, wherein the measurement data comprise electrophysiological measurement data and the at least one sensor comprises at least one electrophysiological sensor.

17. The method of claim 15, wherein the measurement data comprise temperature data and the at least one sensor comprises at least one temperature sensor.

18. The method of claim 13, further comprising arranging a transcranial implant adjacent the tumor resection margin, wherein the transcranial implant houses the light source.
